(19) United States
(12) Patent Application Publication
(10) Pub. No.: US 2016/0123937 A1
(43) Pub. Date: May 5, 2016

(54) METHOD FOR EVALUATING PRETREATMENT OF COAT MATERIAL ON MICRO-PORE OF AUTOMOBILE BODY PANELS

(71) Applicant: Hyundai Motor Company, Seoul (KR)

(72) Inventor: Seung Yul YOON, Seoul (KR)

(73) Assignee: HYUNDAI MOTOR COMPANY

(21) Appl. No.: 14/876,691

(22) Filed: Oct. 6, 2015

(30) Foreign Application Priority Data
Oct. 30, 2014 (KR) 10-2014-0149482

(51) Int. Cl. G01N 33/00

(52) U.S. Cl. CPC G01N 33/00; G01N 2033/0083

(57) ABSTRACT

A method for evaluating pretreatment of a coat material applied to a micro-pore of body panels of an automobile includes preparing a jig including a pair of steel plates, a spacer constituting the micro-pore between the steel plates, and a fixing means fixing the steel plates and the spacer outside the steel plates in order to fix the steel plates and the spacer while the spacer is inserted between the steel plates, inserting the spacer between the steel plates by setting the micro-pore of the jig and manufacturing an evaluating jig by using fixing means, forming a coat by applying the coat material to the micro-pore of the jig, and removing the jig and measuring and evaluating the weight of the formed coat.

### CROSS-REFERENCE TO RELATED APPLICATION

This application claims, under 35 U.S.C. §119(a), the benefit of Korean Patent Application No. 10-2014-0149482, filed on Oct. 30, 2014 with the Korean Intellectual Property Office, the entire contents of which are incorporated herein by reference.

### TECHNICAL FIELD

The present disclosure relates to a method for evaluating pretreatment of a coat material on a micro-pore of body panels of an automobile. More particularly, the present disclosure relates to a method for evaluating pretreatment of a coat material easily applied to a micro-pore of body panels of an automobile without cutting a vehicle application part.

### BACKGROUND

Various methods including chemical conversion pretreatment, electrodeposition and sealers/waxes have been adopted to prevent corrosion of a vehicle body. Among them, the chemical conversion pretreatment method is a method adopted for preventing the corrosion of the vehicle body and securing adhesion with the electrodeposition and pretreating panels of the vehicle body by using the chemical conversion pretreatment as one of the primary processes.

Pretreatment of the vehicle body assembled in a vehicle body manufacturing process includes a de-greasing process, a surface adjusting process, and a chemical conversion coating process in a pretreatment process. During this process, as a material of the chemical conversion coating process, a zinc phosphate based coat material is generally used and in recent years, a zirconium based coating material has been used as an environment-friendly and cost-saving process in some automobile companies.

It is known that the zirconium based coat material is excellent in responsiveness relative to the existing zinc phosphate based coat material, and as a result, the surface adjusting process is not required. Further, the coat is well-formed even at a room temperature within a short time. This characteristic is regarded as one of the advantages of the zirconium based coat material. Therefore, it is predicted that the zirconium based coat material is better in pretreatment than the zinc phosphate based coat material even in a micro-pore such as a vehicle body panel overlapped portion or a tail gate in-outer panel overlapped portion.

However, there has been no evaluation method that can experimentally evaluate the characteristics at present. Therefore, at present, a test is performed by cutting the panel applied to the vehicle. That is, the pretreatment is compared by cutting the panel overlapped portion in order to compare and determine the pretreatment after pretreating the entirety of the vehicle or parts by applying the zinc phosphate based coat material or the zirconium based coat material. However, in spite of the same portion, the micro-pore of the panel overlapped portion has a large deviation of 50 to 400 μm or more, and as a result, reproducibility of the evaluation deteriorates and since the vehicle or the parts need to be pretreated and cut for one evaluation, efficiency significantly deteriorates according to the determination.

However, there is no alternative, and as a result, it is difficult to evaluate the pretreatment of the coat material.

As a technique for determining the characteristic of a chemical conversion coat in the related art, Japanese Patent Application Laid-Open No. 2012-063275 proposes a method that measures a coating rate of the chemical conversion coat using a specular reflection component reflected on the surface of the steel plate by irradiating light to the surface of the steel plate of the vehicle at an irradiation angle in order to measure a chemical conversion coating rate of the steel plate of the vehicle, in which the chemical conversion coat is coated onto the surface.

Japanese Patent Application Laid-Open No. 2005-140599 proposes a coat evaluating method and a quality evaluating/process monitoring system of the chemical conversion coat that can evaluate the quality of a coat by using an actual product without using a processed sample with respect to a crystalline chemical conversion coat such as the zinc phosphate based coat.

Korean Patent Registration No. 10-1267703 proposes a test sample of which quality can be predicted by evaluating various test items used in evaluating a painting property of the steel plate in a laboratory and a method for evaluating the painting property of the steel plate by using the same. Korean Patent Registration No. 10-1149197 relates to a jig for testing the steel plate of an automobile and proposes a method that shortens a time of a phosphate coat attachment amount measuring test by immersing a plurality of samples in a coat peel-off liquid by using one jig and increases test efficiency and test convenience.

However, since the test and measurement methods for the painted coat in the related art are methods in which the product or parts actually applied to the vehicle are cut or used as described above or a comparison test for evaluating pretreatment of the micro-pore is impossible, a problem in that the test and measurement methods cannot be adopted as the pretreatment comparing and evaluating test for the micro-pore of the body panels of the automobile still exists.

The above information disclosed in this Background section is only for enhancement of understanding of the background of the disclosure and therefore it may contain information that does not form the prior art that is already known in this country to a person of ordinary skill in the art.

### SUMMARY OF THE DISCLOSURE

The present disclosure has been made in an effort to solve the above-described problems associated with prior art and to provide a method for conveniently comparing and evaluating pretreatment of coat materials in a micro-pore of body panels of an automobile through a sample without inconveniences such as cutting, or the like after application to a vehicle or an uneconomical method in comparing and evaluating the pretreatment of the coat materials.

Therefore, the present disclosure has been made in an effort to provide a method that can easily compare and evaluate pretreatment of coat materials by using a jig for evaluating the pretreatment of the coat materials in the micro-pore of the body panels of the automobile.

The present disclosure has also been made in an effort to provide an evaluating jig that can compare and evaluate the pretreatment of the coat materials by similarly configuring application environments to the coat materials applied to the micro-pore of the body panels of the automobile.

In one aspect, the present disclosure provides a method for evaluating pretreatment of a coat material applied to a micro-pore of body panels of an automobile, including: preparing a jig including a pair of steel plates, a spacer constituting the micro-pore between the steel plates, and a fixing means fixing the steel plates and the spacer outside the steel plates in order to fix the steel plates and the spacer while the spacer is inserted between the steel plates; inserting the spacer between the steel plates by setting the micro-pore of the jig and manufacturing an evaluating jig by using fixing means; forming a coat by applying the coat material to the micro-pore of the jig; and removing the jig and measuring and evaluating the weight of the formed coat.

In another aspect, the present disclosure provides a jig having the configuration, which is used in the method for evaluating pretreatment of a coat material applied to a micro-pore of body panels of an automobile.

According to the present disclosure, the number of processes including pretreating and cutting a vehicle or a part can be reduced by separately manufacturing and using an evaluating jig in evaluating pretreatment of a coat material applied to a micro-pore of body panels of an automobile to enable pretreatment evaluation by an economical and efficient method.

Since the evaluation is enabled in various structures by diversifying the shape of the micro-pore according to a configuration of the jig and a reproducible evaluation result can be acquired by setting a constant micro-pore, and developing and standardizing the coat material such as a zirconium based pretreating material, or the like are easy afterwards.

Other aspects and preferred embodiments of the disclosure are discussed infra.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The above and other features of the disclosure are discussed infra.

### BRIEF DESCRIPTION OF THE DRAWINGS

The above and other features of the present disclosure will now be described in detail with reference to certain exemplary embodiments thereof illustrated in the accompanying drawings which are given hereinbelow by way of illustration only, and thus are not limitative of the present disclosure, and wherein:

FIG. 1 is a configuration diagram of an evaluating jig used with a method for evaluating pretreatment of a coat material applied to a micro-pore of body panels of an automobile according to the present disclosure;

FIG. 2 is a schematic process diagram conceptually illustrating a process for evaluating pretreatment of the coat material applied to the micro-pore by using the jig according to the present disclosure;

FIG. 3(a) is a graph illustrating a change in weight of a coat as a result of evaluating pretreatment of each micro-pore for a zinc phosphate based coat material by using the evaluating jig in an embodiment of the present disclosure; and

FIG. 3(b) is a graph illustrating a change in weight of a coat as a result of evaluating pretreatment of each micro-pore for a zirconium based coat material by using the evaluating jig in an embodiment of the present disclosure; and

FIG. 4 is a SEM photograph for each micro-pore acquired by observing coat states of the zinc phosphate based coat material and the zirconium based coat material by using the evaluating jig according to the embodiment of the present disclosure.

It should be understood that the appended drawings are not necessarily to scale, presenting a somewhat simplified representation of various preferred features illustrative of the basic principles of the disclosure. The specific design features of the present disclosure as disclosed herein, including, for example, specific dimensions, orientations, locations, and shapes will be determined in part by the particular intended application and use environment.

In the figures, reference numbers refer to the same or equivalent parts of the present disclosure throughout the several figures of the drawing.

### DETAILED DESCRIPTION

Hereinafter reference will now be made in detail to various embodiments of the present disclosure, examples of which are illustrated in the accompanying drawings and described below. While the disclosure will be described in conjunction with exemplary embodiments, it will be understood that present description is not intended to limit the disclosure to those exemplary embodiments. On the contrary, the disclosure is intended to cover not only the exemplary embodiments, but also various alternatives, modifications, equivalents and other embodiments, which may be included within the spirit and scope of the disclosure as defined by the appended claims.

Hereinafter, the present disclosure will be described below in more detail as one implementation embodiment.

The present disclosure proposes a method of setting a micro-pore by using an evaluating jig that may compare and evaluate pretreatment of the micro-pore of body panels of an automobile with respect to the existing zinc phosphate based coat material and zirconium based coat material applied to the body panels of the automobile, applying a coat material to the set micro-pore, and removing and disassembling the jig to evaluate the pretreatment.

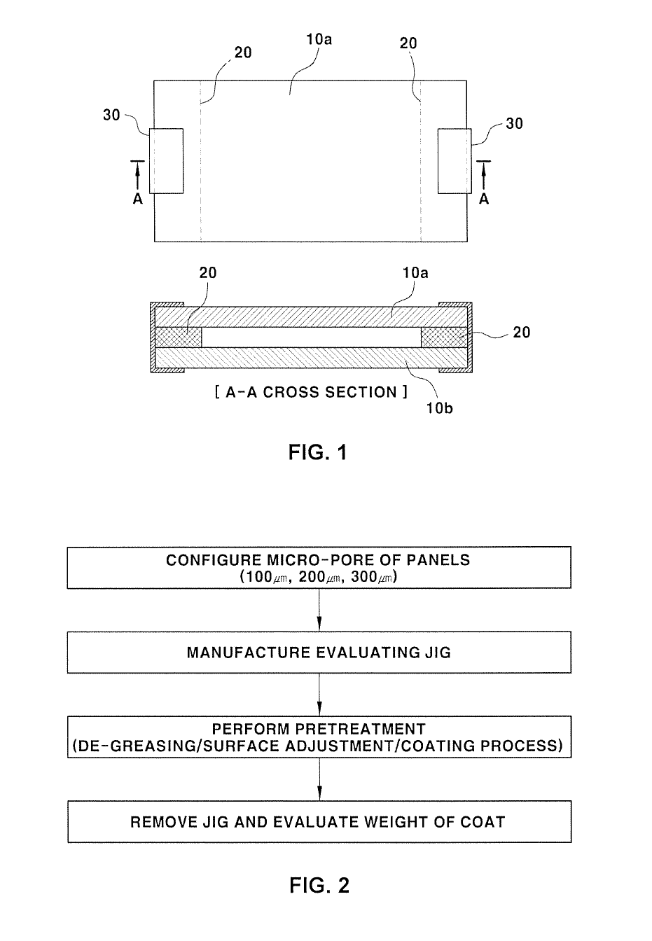

In the method for evaluating the pretreatment of the coat material according to the present disclosure, an evaluating jig is used. A preferred implementation embodiment of the evaluating jig according to the present disclosure is illustrated in FIG. 1.

In the pretreatment evaluating method using the evaluating jig, the pretreatment may be evaluated like a process diagram illustrated in FIG. 2.

Referring to FIG. 1, the pretreatment evaluating jig according to the present disclosure includes a pair of steel plates 10a and 10b, a spacer 20 constituting the micro-pore between the steel plates 10a and 10b, and a fixing means 30 fixing the steel plates 10a and 10b and the spacer 20 outside the steel plates 10a and 10b in order to fix the steel plates 10a and 10b and the spacer 20 while the spacer 20 is inserted between the steel plates 10a and 10b.

According to a preferred implementation embodiment of the present disclosure, the spacer 20 is inserted between the steel plates 10a and 10b by configuring the micro-pore of the jig with a desired width (interval) after preparing the evaluating jig, and the evaluating jig is manufactured by using a fixing means 30. In this case, one or two or more spacers 20 may be overlapped and used. Further, since the fixing means 30 does not perform a function other than fixing the steel plate and the spacer 20, a nal-clip, or the like, may be generally used.

According to the present disclosure, when the evaluating jig is manufactured, the width (interval) of the micro-pore between the steel plates 10a and 10b may be controlled. Since the width of the micro-pore may be determined according to the thickness of the spacer 20 between the steel plates 10a and 10b, one or several spacers 20 are overlapped to control the micro-pore.

According to the preferred implement embodiment of the present disclosure, the steel plate may be manufactured and used with a size of 3×3 cm to 5×5 cm, that is, a size of (3 to 5)×(3 to 5) cm. According to the present disclosure, since the weight of the coat is measured by using table-type XRF equipment in order to minimize an error at the time of measuring the weight of the coat after pretreating the zirconium based coat material in relation with the size of the steel plate, the steel plate may be manufactured and used with a size suitable for the measurement and the measurement may be enabled only when a sample is in the minimum range. Further, as the sample increases in size, a measurement error decreases, but it is difficult to constantly maintain the micro-pore due to bending of the steel plate. The size of the steel plate is preferably a maximum of 5×5 cm when considering a space occupied by the spacer in a steel plate area into which the spacer may be inserted and an open space maintaining the micro-pore. As the material is steel plate, the same material as that applied to the body panels of the automobile may be used.

According to the preferred implementation embodiment of the present disclosure, the spacer may comprise, a fluoroplastic-based resin which is excellent in chemical resistance such as acid resistance, alkali resistance, or the like, may be used. For example, PTFE may be used. In addition to the spacer, a film having a thickness of approximately 100 μm is used to prevent a pretreating liquid from being contaminated due to chemical decomposition of the spacer during de-greasing and chemical conversion coat processes. The size of the spacer used in the present disclosure may be set with an appropriate size according to the size of the steel plate.

According to the preferred implementation embodiment of the present disclosure, since one or more spacers are overlapped at the time of manufacturing the evaluating jig, an evaluating jig may be manufactured in which the micro-pore of the steel plate has a size of 100 μm by using one space having the thickness of 100 μm, the micro-pore has a size of 200 μm by using two spacers which are overlapped, and the micro-pore has a size of 300 μm by using three spacers which are overlapped.

According to the preferred implementation embodiment of the present disclosure, the jig is preferably manufactured so that the pore between the steel plates becomes 100 to 400 μm. When the micro-pore between the steel plates is less than 100 μm, there is a problem that the coat is not formed and when the pore between the steel plates is wider than 400 μm, the coat is normally formed and the micro-pore is too wide and as a result, there is no difference in coat forming states between materials of the pretreating coat material after coating the pretreated coat material. Therefore, there is no discrimination, and as a result, it is difficult to evaluate the pretreatment.

According to the present disclosure, after manufacturing the evaluating jig, a coat forming step of coating and applying the coat material to the micro-pore of the jig is performed.

According to the preferred implementation embodiment of the present disclosure, a de-greasing process may be additionally performed before coating the coat material as the pretreatment. This is a process for removing impurities on the surface of the steel plate of the evaluating jig. When the de-greasing process is performed, the performance of the pretreating coat material may be more accurately evaluated.

According to the preferred implementation embodiment of the present disclosure, after the de-greasing process is performed before coating the coat material, a surface adjusting process may be additionally performed. The surface adjusting process is applied to form a uniform particle size and a compact coat crystal at the time of coating the coat material and is required only when the zinc phosphate based coat material is applied. Therefore, the surface adjusting process is not required when the zirconium based coat material is applied.

According to the preferred implementation embodiment of the present disclosure, after the de-greasing process or the de-greasing process and the surface adjusting process in some cases are additionally performed, the pretreating coat material is coated and applied to the evaluating jig through the coating process to form the pretreated coat.

After forming the coat in the evaluating jig, the evaluating jig is removed and the weight of the formed coat is measured and evaluated.

Herein, after forming the coat in the evaluating jig, the evaluating jig is removed and the weight of the coat formed on an inner surface of the micro-pore is measured. Thereafter, an inner surface area of the micro-pore is calculated to evaluate a weight per unit area.

According to the present disclosure, a generally well-known method may be used to evaluate whether the pretreated coat is formed. According to the preferred implementation embodiment of the present disclosure, when the pretreated coat is the zinc phosphate based coat, the weight of the coat may be measured by using a chromic acid anhydride method and a particle size may be verified by SEM. Further, when the pretreated coat is the zirconium based coat, the weight of the coat may be measured by using table-type XRF and since the zirconium based coat is noncrystallin, verifying the particle size is not required.

As described above, the method for evaluating the pretreatment of the coat according to the present disclosure is manufacturing an evaluating jig simulating the micro-pore of the body panels of the automobile and performing evaluations and using the evaluating jig.

Therefore, the present disclosure includes a jig for evaluating the pretreatment of the coat material applied to the micro-pore of the body panels of the automobile.

In the present disclosure, since the evaluation may be performed by using the evaluating jig simulating the micro-pore of the panels under the same condition as the body panels of the automobile applied to the vehicle, there is no inconvenience that the pretreatment is compared by cutting a panel overlapped portion after pretreating the entirety of the vehicle or the part during the existing evaluation. Further, since the micro-pore of the evaluating jig is easily controlled, reproducibility of evaluation considering various micro-pores is excellent and an efficiency of an evaluation test is also excellent.

Therefore, according to the present disclosure, a simple, convenient, and economical evaluation method which may compare and evaluate the pretreatment of the micro-pores of the body panels of an automobile between the existing zinc phosphate based coat material and the zirconium based coat material may significantly contribute to improving the quality of the automobile. In particular, it is expected that completion of the development of the zirconium based pretreating material will also contribute to standardization regarding the pretreated coat material applied to the body panels of an automobile.

The evaluating jig of the present disclosure or the evaluation method using the same may be variously applied to verify a painting pretreating state of various industrial apparatuses in addition to the body panels of an automobile.

Hereinafter, although the present disclosure is described in detail according to the embodiment, the present disclosure is not limited to the embodiment.

### EXAMPLE

The pretreatment evaluating jig having the configuration illustrated in FIG. 1 was manufactured and specifications for manufacturing the jig were prepared under a condition shown in Table 1 to perform the pretreatment and the evaluation.

TABLE 1

| | | | size of | treatment | |
|---|---|---|---|---|---|
| classification | pretreatment material | steel plate | steel plate | condition | pore of panels |
| contents | zinc phosphate based, | CR steel plate | 5 × 4 cm | 35° C. × 90 sec | 100 μm, 200 μm, |
| | zirconium based | | | | 300 μm |

The pretreated coat was applied to the evaluating jig under the test condition and the pretreated coat was removed and released. Thereafter, the weight of a sample was measured before removing the coat in the case of the zinc phosphate based coat and the weight of the sample was measured after removing the coat by the chromic acid anhydride method to create a difference therebetween as the weight of the coat and the surface area of the sample was calculated to acquire a coat weight (g/m²). Further, in the case of the zirconium based coat, the weight of Zr was measured by using the table-type XRF equipment. Thereafter, the surface area of the sample was calculated to acquire the coat weight (mg/m²). The result thereof is shown in Table 2 below.

TABLE 2

| classification | | zinc phosphate based | | | zirconium based | | |
|---|---|---|---|---|---|---|---|
| micro-pore | | 100 μm | 200 μm | 300 μm | 100 μm | 200 μm | 300 μm |
| weight of | measurement value | 1.00 g/m² | 1.39 g/m² | 1.62 g/m² | 3.47 mg/m² | 19.31 mg/m² | 26.92 mg/m² |
| coat | amount of coat | 55.3% | 77.2% | 90.2% | 51.9% | 96.6% | 134.6% |
| | compared to | | | | | | |
| | specification | | | | | | |
| | (%) | | | | | | |

As shown in Table 2, according to the result of measuring the coat weight by using the evaluating jig to compare the pretreatment of the micro-pore of the body panels of an automobile, it may be quantitatively verified that micro-pore pretreatment of zirconium based pretreatment was better than the zinc phosphate based pretreatment for each micro-pore.

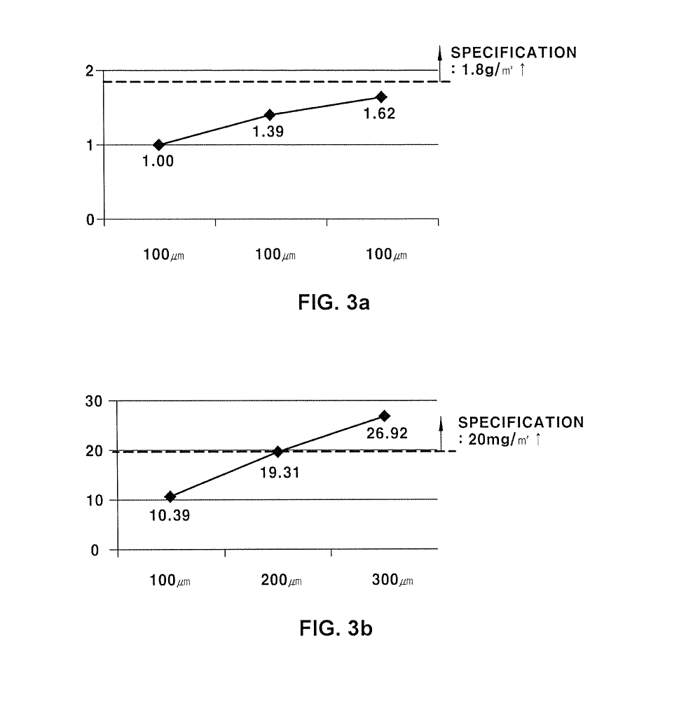

In order to compare quantitative evaluation contents of Table 2, the quantitative evaluation contents are shown in a graph and illustrated in FIG. 3. FIG. 3 is a graph illustrating a change in weight of a coat as a result of evaluating pretreatment of each micro-pore for a zinc phosphate based coat material (a) and a zirconium based coat material (b) by using the evaluating jig in an embodiment of the present disclosure.

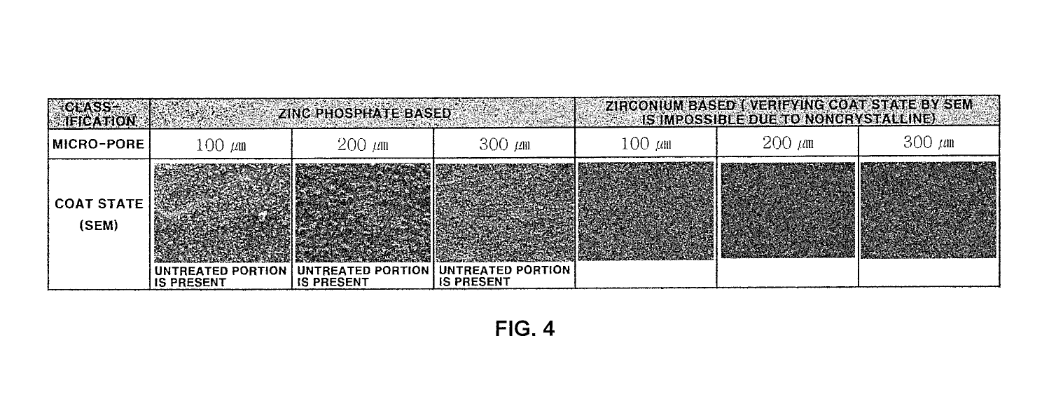

Meanwhile, a result of an SEM photograph of each coat is illustrated in FIG. 4 to evaluate the pretreatment of the coat state for the micro-pore of the coat acquired through each evaluating jig.

Referring to a comparison photograph of FIG. 4, in the case of the zinc phosphate based coat, the coat state in the micro-pore regarding how many untreated portions are generated for each micro-pore may be clearly verified to enable accurate evaluation. However, since the zirconium based coat material is noncrystalline, the coat state may not be verified through the SEM.

The pretreatment of the coat of the micro-pore of the body panels of the automobile according to the present disclosure is very useful in economically and conveniently evaluating the pretreatment of the body panels of the automobile without cutting the parts applied to the vehicle, because it is very easy and simple to evaluate the coat state by using the evaluating jig.

The method for evaluating pretreatment of the coat of the micro-pore of the body panels of the automobile according to the present disclosure can be applied to verify a painting pretreatment state of various industrial apparatuses.

The disclosure has been described in detail with reference to preferred embodiments thereof. However, it will be appreciated by those skilled in the art that changes may be made in these embodiments without departing from the principles and spirit of the disclosure, the scope of which is defined in the appended claims and their equivalents.

What is claimed is:

1. A method for evaluating pretreatment of a coat material applied to a micro-pore of body panels of an automobile, the method comprising:
preparing a jig including a pair of steel plates, a spacer constituting the micro-pore between the steel plates, and a fixing means fixing the steel plates and the spacer outside the steel plates in order to fix the steel plates and the spacer while the spacer is inserted between the steel plates;
inserting the spacer between the steel plates by setting the micro-pore of the jig and manufacturing an evaluating jig by using fixing means;
forming a coat by applying the coat material to the micro-pore of the jig; and
removing the jig and measuring and evaluating the weight of the formed coat.

2. The method of claim 1, wherein the steel plate is manufactured with a size of 3×3 to 5×5 cm.

3. The method of claim 1, wherein the manufacturing of the evaluating jig includes controlling the micro-pore by overlapping one spacer.

4. The method of claim 1, wherein the manufacturing of the evaluating jig includes controlling the micro-pore by overlapping a plurality of spacers.

5. The method of claim 1, wherein the jig is manufactured so that the micro-pore between the steel plates becomes 100 to 400 μm.

6. The method of claim 1, wherein a de-greasing process is performed before forming the coat.

7. The method of claim 1, wherein a surface adjusting process is performed before forming the coat.

8. A jig for evaluating pretreatment of a coat material applied to a micro-pore of body panels of an automobile, the jig comprising:
a pair of steel plates;
a spacer constituting the micro-pore between the steel plates; and
a fixing means fixing the steel plates and the spacer outside the steel plates in order to fix the steel plates and the spacer while the spacer is inserted between the steel plates.

9. The jig of claim 8, wherein the size of the steel plate is 3×3 to 5×5 cm.

10. The jig of claim 8, wherein the spacer has a thickness of 100 to 400 μm.

11. The jig of claim 8, wherein one spacer is included.

12. The jig of claim 8, wherein a plurality of spacers are included.
